(19) United States
(12) Patent Application Publication
(10) Pub. No.: US 2010/0130462 A1
(43) Pub. Date: May 27, 2010

(54) LANOLIN COMPOSITIONS AND METHODS FOR MAKING AND USING SAME

(76) Inventors: Sherry L. Thomas, Hanover, MA (US); Laura Marseglia, Newton, MA (US); Stephen F. Hanson, North Attleboro, MA (US); Mohsen Amiran, Oak Creek, WI (US)

Correspondence Address: MICHAEL BEST & FRIEDRICH LLP, Two Prudential Plaza, 180 North Stetson Avenue, Suite 2000, CHICAGO, IL, 60601, US

(21) Appl. No.: 12/596,334

(22) PCT Filed: Apr. 18, 2008
(86) PCT No.: PCT/US08/60890

§ 371 Date: Oct. 16, 2009

Related U.S. Application Data
(60) Provisional application No. 60/913,238, filed on Apr. 20, 2007.
(60) Provisional application No. 60/913,490, filed on Apr. 23, 2007.

(51) Int. Cl. A61K 31/56; A61P 43/00

(52) U.S. Cl. 514/182

(57) ABSTRACT

A composition suitable for application to the skin comprising lanolin, an oil, an anti-oxidant, and a combination thereof. The composition is suitable for particular application to a human breast.

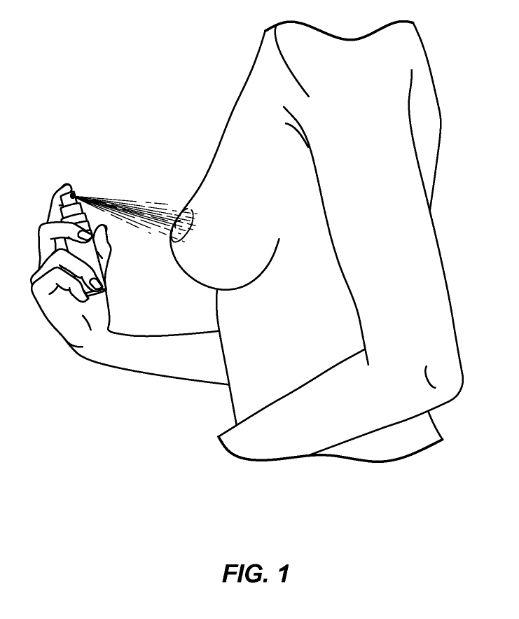

### RELATED APPLICATIONS

This application claims priority to provisional patent application Nos. 60/913,238 and 60/913,490, filed Apr. 20, 2007 and Apr. 23, 2007, respectively, the entire contents of which are incorporated herein by reference.

### BACKGROUND OF THE INVENTION

The use of creams, ointments, and lotions is known in the art to soothe dry, cracked skin, for example, breasts or nipples while a woman is breastfeeding. However, the application of a cream, ointment, or lotion may be painful. Additionally, the presence of certain ingredients, for example, C1-C4 alcohols, tends to render the application of such compositions unpleasant.

### SUMMARY OF THE INVENTION

The invention provides lanolin liquid composition and a way to apply the lanolin liquid onto human skin, particularly breasts or nipples to soothe dry, cracked breasts or nipples.

Described below are lanolin liquid compositions for use in treating skin, particularly breasts and/or nipples, methods for making the lanolin liquid compositions, and methods for applying the lanolin liquid compositions. The lanolin liquid composition is typically made from lanolin oil, sunflower seed oil, caprylic/capric triglycerides, and vitamin E acetate. The lanolin liquid composition has characteristics that render it suitable for spraying directly onto the skin.

Other aspects of the invention will become apparent by consideration of the detailed description and accompanying drawing.

### BRIEF DESCRIPTION OF THE DRAWING

The figure illustrates a woman spraying a lanolin liquid composition on her breast.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It also is understood that any numerical range recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this application.

In one embodiment, the invention provides a lanolin liquid composition comprising at least lanolin oil. The lanolin liquid composition may also comprise oils, skin protectants, humectants, emollients, skin conditioning agents, anti-oxidants, or combinations thereof. In one embodiment, the lanolin liquid composition may comprise at least one of lanolin, sunflower seed oil, caprylic/capric triglycerides, and vitamin E acetate.

As used herein, the term “breast” means a female, human breast including the nipple and areola.

As used herein, the term “nipple” means a projection at the apex of the breast on the surface of which the lactiferous ducts open.

As used herein, the term “areola” means the circular, pigmented area surrounding the nipple.

Lanolin oil may include, but is not limited to, lanolin, or pure lanolin (lansinoh). The lanolin liquid composition may comprise lanolin, lanolin oil, or a combination thereof. Lanolin is a waxy secretion of the ovine sebaceous gland that consists primarily of various esters that include compounds exhibiting a wide range of viscosity from mobile liquids to hard waxes. Lanolin has long been a constituent of hand and nail creams. Lanolin oil is a physical derivative of lanolin typically produced by subjecting lanolin to low temperature fractional crystallization to isolate those liquid esters of conventional anhydrous lanolin that are liquid at room temperature (about 25° C.). Lanolin oil, accordingly, exhibits a greatly reduced viscosity relative to lanolin and can be used in formulations to provide certain of the benefits of lanolin while providing a lighter and less viscous composition. Although different with regard to many physical properties, the lighter esters of lanolin present in lanolin oil are similar in their chemical and general properties to whole lanolin and tend to retain and exhibit the high emollient power associated with lanolin.

In one embodiment, lanolin and lanolin oil may both be used in the lanolin liquid composition. Mixing lanolin oil with lanolin may produce a composition exhibiting a reduced melting point, reduced viscosity, and reduced drag on the skin. However, although lanolin, and mixtures of lanolin with lanolin oil, are absorbed by the human stratum corneum, the rate of absorption is relatively slow even when assisted by rubbing and, at loadings in excess of 2 mg/cm², absorption may be incomplete and may produce a thin greasy film on the skin surface.

The lanolin liquid composition may also include, without limitation, a skin conditioning agent. Such an agent may comprise a substance that enhances the appearance of dry or damaged skin, as well as a material that may adhere to the skin to reduce flaking, restore suppleness, and generally improve the appearance of skin. Specific examples of skin conditioning agents include, without limitation, acetyl cysteine, N-acetyl dihydrosphingosine, acrylates/behenyl acrylate/dimethicone acrylate copolymer, adenosine, adenosine cyclic phosphate, adenosine phosphate, adenosine triphosphate, alanine, albumen, algae extract, allantoin and derivatives, aloe barbadensis extracts, amyloglucosidase, arbutin, arginine, bromelain, buttermilk powder, butylene glycol, calcium gluconate, carbocysteine, carnosine, beta-carotene, casein, catalase, cephalins, ceramides, chamomilla recutita (matricaria) flower extract, cholecalciferol, cholesteryl esters, coco-betaine, corn starch modified, crystallins, cycloethoxymethicone, cysteine DNA, cytochrome C, darutoside, dextran sulfate, dimethicone copolyols, dimethylsilanol hyaluronate, elastin, elastin amino acids, ergocalciferol, ergosterol, fibronectin, folic acid, gelatin, gliadin, beta-glucan, glucose, glycine, glycogen, glycolipids, glycoproteins, glycosaminoglycans, glycosphingolipids, horseradish peroxidase, hydrogenated proteins, hydrolyzed proteins, jojoba oil, keratin, keratin amino acids, kinetin, lactoferrin, lanosterol, lecithin, linoleic acid, linolenic acid, lipase, lysine, lysozyme, malt extract, maltodextrin, melanin, methionine, niacin, niacinamide, oat amino acids, oryzanol, palmitoyl hydrolyzed proteins, pancreatin, papain, polyethylene glycol, pepsin, phospholipids, phytosterols, placental enzymes, placental lipids, pyridoxal 5-phosphate, quercetin, resorcinol acetate, riboflavin, saccharomyces lysate extract, silk amino acids, sphingolipids, stearamidopropyl betaine, stearyl palmitate, tocopherol, tocopheryl acetate, tocopheryl linoleate, ubiquinone, vitis vinifera (grape) seed oil, wheat amino acids, xanthan gum, zinc gluconate, or combinations thereof. A skin conditioning agent, other than those listed above, may also be used, as is readily appreciated by those skilled in the art.

The lanolin liquid composition may also include a skin protectant, which may protect injured or exposed skin or mucous membrane surfaces from harmful or irritating external compounds. Specific examples of skin protectants include, without limitation, algae extract, allantoin, aluminum hydroxide, aluminum sulfate, camellia sinensis leaf extract, cerebrosides, dimethicone, glucuronolactone, glycerin, kaolin, lanolin, malt extract, mineral oil, petrolatum, potassium gluconate, talc, or combinations thereof. Those skilled in the art will readily appreciate that a skin protectant, other than those listed above, may be included in the compositions used.

An emollient is an ingredient that may help skin maintain a soft, smooth, and pliable appearance. Specific examples of emollients include, without limitation, acetyl arginine, acetylated lanolin, algae extract, apricot kernel oil polyethylene glycol-6 esters, avocado oil polyethylene glycol-11 esters, bis-polyethylene glycol-4 dimethicone, butoxyethyl stearate, C₁₈ C₃₆ acid glycol ester, C₁₂ C₁₃ alkyl lactate, caprylyl glycol, cetyl esters, cetyl laurate, coconut oil polyethylene glycol-10 esters, di-C₁₂ C₁₃ alkyl tartrate, diethyl sebacate, dihydrocholesteryl butyrate, dimethiconol, dimyristyl tartrate, disteareth-5 lauroyl glutamate, ethyl avocadate, ethylhexyl myristate, glyceryl isostearates, glyceryl oleate, hexyldecyl stearate, hexyl isostearate, hydrogenated palm glycerides, hydrogenated soy glycerides, hydrogenated tallow glycerides, isostearyl neopentanoate, isostearyl palmitate, isotridecyl isononanoate, laureth-2 acetate, lauryl polyglyceryl-6 cetearyl glycol ether, methyl gluceth-20 benzoate, mineral oil, myreth-3 palmitate, octyldecanol, octyldodecanol, odontella aurita oil, 2-oleamido-1,3 octadecanediol, palm glycerides, polyethylene glycol avocado glycerides, polyethylene glycol castor oil, polyethylene glycol-22/dodecyl glycol copolymer, polyethylene glycol shea butter glycerides, phytol, raffinose, stearyl citrate, sunflower seed oil glycerides, tocopheryl glucoside, or combinations thereof. Those skilled in the art will readily appreciate that emollients, other than those listed above, may also be used.

Humectants are ingredients that may help maintain moisture levels in skin. Specific examples of humectants include, without limitation, acetyl arginine, algae extract, aloe barbadensis leaf extract, 2,3-butanediol, chitosan lauroyl glycinate, diglycereth-7 malate, diglycerin, diglycol guanidine succinate, erythritol, fructose, glucose, glycerin, honey, hydrolyzed wheat protein/polyethylene glycol-20 acetate copolymer, hydroxypropyltrimonium hyaluronate, inositol, lactitol, maltitol, maltose, mannitol, mannose, methoxy polyethylene glycol, myristamidobutyl guanidine acetate, polyglyceryl sorbitol, potassium PCA, propylene glycol, sodium PCA, sorbitol, sucrose, urea, or combinations thereof. Other humectants may be used for yet additional embodiments of this invention, as will be appreciated by those skilled in the art.

The lanolin liquid composition may also include, without limitation, oils. Oils may include, without limitation, ester oils. Examples of specific oils include, but are not limited to, vegetable oils. Examples of vegetable oils may include, without limitation, sunflower seed oil or linoleic acid, canola oil, safflower oil, noni seed oil, corn oil, soy oil, evening primrose oil, pumpkin oil, wheat germ oil, rice bran oil, fractionated coconut oil or caprylic/capric triglycerides, and palm kernel oil, or combinations thereof.

Vegetable oils may serve as a solvent and viscosity modifier for the lanolin oil. In addition to reducing the viscosity of the composition, vegetable oil may improve the adsorption of the lanolin oil into the treated skin and/or serve as a source of linoleic acid for the composition. Linoleic acid is a polyunsaturated fatty acid, specifically an omega-6 fatty acid, found in the lipids of cell membranes. Linoleic acid is one of a number of fatty acids that form an essential dietary requirement for all mammals. Omega-6 fatty acid deficiencies may lead to symptoms including, for example, dry hair, hair loss, and poor wound healing. Linoleic acid is used in making soaps, emulsifiers, and quick-drying oils, and has become increasingly popular in the personal products industry as a result of its attributed beneficial properties, including, for example, anti-inflammatory, acne reduction, and moisture retention properties.

The lanolin liquid composition may also include, without limitation, one or more triglyceride derivatives, such as caprylic or capric triglyceride. Triglycerides may serve as an emollient that may, and possibly in combination with oils, serve to increase the penetration and absorption of the lanolin oil, thereby reducing or eliminating the greasy film on the skin surface.

The lanolin liquid composition may also include, without limitation, anti-oxidants and skin conditioning agents. Anti-oxidants and skin conditioning agents may include, but are not limited to, tocopherols and derivatives. Examples of tocopherols and derivates may include, without limitation, vitamin E acetate. Additional examples of anti-oxidants may include vitamin anti-oxidants and vitamin C and its derivatives.

The lanolin liquid composition may also comprise various skin care ingredients, including, without limitation, allantoin, beeswax, castor oil, citric acid, colloidal oatmeal, decyl glucoside, Di-PPG-2 myreth-10 adipate, disodium cocoamphodiacetate, glycerin, green tea extract, jojoba oil, lecithin, meadowfoam oil, olive butter, PEG 150 pentaerythrityl tetrastearate, propylene glycol, diazolidinyl urea, iodopropynyl butylcarbamate, shea butter, silk powder, sodium hydromethylglycinate, sodium PCA, squalane, tapioca starch, or combinations thereof.

The lanolin liquid composition may further comprise cosmetically active substances. As used herein, the term “cosmetically active substances” means both compounds and mixtures of compounds suitable for topical administration to produce one or more beneficial effects on the condition or appearance of the treated skin. Such active substances may produce both pharmacological and cosmetic effects for varying periods of time and will typically include such active substances as, for example, emollients, humectants, vitamins, hormones, sunscreens, antimicrobials, antiperspirants, deodorants, anesthetics, and other therapeutic substances. The cosmetically active substances may be present in the lanolin liquid composition in amounts varying from 0% to 95% by weight of the final composition, particularly from about 0.1% to about 95% by weight of the final composition, and more particularly from about 0.5% to about 50% by weight of the final composition.

The lanolin liquid composition may further comprise one or more surface active substances (surfactants). Surfactants may increase the stability of the composition. Without being limited by theory, it is believed that by suppressing or reducing coalescence in which small droplets recombine to form larger droplets and/or creaming in which one of the substances comprising the emulsion tends to migrate to the top surface of the emulsion under the influence of buoyancy, stability may increase.

The compositions of the present application may be prepared using different methods. In one embodiment, the lanolin liquid composition may go through multiple filtration stages followed by distillation, or molecular distillation. In a further embodiment, the lanolin liquid composition is prepared by distilling raw farmed lanolin. The resulting distilled product is then filtered with 3× filtration, followed by molecular distillation (steam). This step is followed by retaining the light oil fraction and removing waxes, acids, heavy oils, and other undesirable components. This light oil fraction is finally diluted with oils. In some embodiments, the lanolin liquid composition may be substantially free of waxes, heavier oils, acids, wool acids, or combinations thereof. Without being limited by theory, waxes, heavy oils, acids, and wool acids may have a greater chemical affinity for pesticides. Without being limited by theory, it may be the removal of these waxes, heavy oils, and acids which may result in lower pesticide levels. The process may extract heavier compounds and dilute the lanolin with known clean organic, natural, and pesticide-free ingredients, which may result in substantially purer compositions. In some embodiments, the lanolin liquid composition may be prepared aseptically.

The amount of lanolin oil (by weight) in the composition may be from about 1% to about 60%, particularly from about 10% to about 60%, particularly from about 1% to about 30%, more particularly about 10%.

The amount of oil (by weight) in the composition may be from about 1% to about 75%, particularly from about 20% to about 50%, and more particularly about 25%.

The amount of sunflower seed oil (by weight) in the composition may be from about 1% to about 75%, more particularly about 25%.

The amount of caprylic/capric triglycerides (by weight) in the composition may be from about 1% to about 90%, particularly from about 30% to about 70%, and more particularly about 64.9%.

The amount of tocopherol and derivatives (by weight) in the composition may be from about 0.01% to about 5%, particularly from about 0.1% to about 3%, and more particularly about 0.1%.

Although some references suggest that mineral oil is an acceptable substitute for lanolin oil in any type of skin care oil or formulation, lanolin oil tends to exhibit improved emollient performance and increase protection from epidermal moisture loss. Lanolin oil also has attributes that render it useful for treating irritated tissues, including, for example, breast tissue, in that it may maintain increased humidity at and near the skin surface, and allows for gas exchange. The lanolin liquid composition of the present invention is non-irritating and non-sensitizing to the skin, hypoallergenic, non-irritating to the eye, non-irritating to the mucous membranes, and non-toxic if ingested. Moreover, lanolin oil does not have a strong flavor or odor that would tend to discourage suckling by an infant. In some embodiments, the lanolin liquid composition may be all-natural or organic, contain no preservatives, or combinations thereof. In further embodiments, the lanolin liquid composition may be substantially or completely lipophilic (made with oils) and incapable of sustaining microbial life. The lanolin liquid composition may possess ingredients which are pharmaceutical grade or higher. The lanolin liquid composition may be refrigerated to provide cooling comfort upon application and/or improve the stability of the composition. The lanolin liquid composition has a light viscosity and may not solidify when cooled. Lanolin oil may have an optimal effectiveness at about 5-15%. Lanolin oil may provide a moisture retention efficacy at approximately 15%. Lanolin may have better effectiveness at creating a moisture shield as compared to petrolatum products. The lanolin liquid composition may, without limitation, speed skin repair, increase skin moisture, prevent moisture loss, reduce the risk of skin infections, maintain clear skin pores, maintain clear milk ducts, increase skin elasticity, reduce the appearance of stretch marks, soothe and restore skin health, or combinations thereof. The lanolin liquid composition may be safe for infants. The lanolin liquid composition may use oils that have nutritional value.

A method for applying the lanolin liquid composition may be employed. In one embodiment, the application of the composition is accomplished with the use of spraying technology. The lanolin liquid composition can be produced to have characteristics that make it suitable for spraying onto the skin. Formulations of the present invention may have ingredients and properties (e.g., viscosity, surface tensions, solubilities, drying times, etc.) that may make them suitable for use with sprayers and/or misters. Any type of sprayer and/or mister is suitable for use with the present invention. In another embodiment, the lanolin liquid composition may be provided in a container configured for delivering the composition to the skin while reducing the level of contact associated with the application of the composition to the skin or tissue.

In a further embodiment, the lanolin liquid composition may be provided in a range of viscosities whereby the viscosity of the particular composition or range of compositions may be selected to correspond to the delivery method utilized. As will be appreciated by those skilled in the art, for example, those compositions intended for application with a pump sprayer may be somewhat less viscous than compositions intended for aerosol delivery using a propellant. A range of delivery systems may be utilized for delivering compositions of the present invention to an affected region. For instance, in an aerosol delivery system, the lanolin liquid composition may be utilized with a range of application systems including those in which a propellant and the composition are combined in a single chamber and those in which a propellant is isolated from the composition by a membrane or other barrier.

The lanolin liquid composition may be applied to any area of the skin, particularly, to the breasts, and more particularly to the areola and nipples.

In one embodiment, the lanolin liquid composition may be applied by spraying directly onto a breast, areola, nipple, or a combination thereof, as illustrated in the Figure. The lanolin liquid composition may be applied by holding a sprayer in a hand, with a finger on a nozzle. In one embodiment, the sprayer is held approximately 6-12 inches from the nipple, the nozzle may be pointed at a somewhat perpendicular angle to the nipple, with an opening of the nozzle facing the nipple, and the nozzle is compressed by pushing down with the finger. In other embodiments, the sprayer may be held at any distance from the nipple. In a further embodiment, the lanolin liquid may be sprayed on a finger and applied to the skin by rubbing with the finger.

In some applications, a sprayer is configured to apply a lanolin liquid composition with a round shape characterized by having a spot size from about 1 inch to about 3 inches in diameter. The sprayer may include an adjustable nozzle that can apply a lanolin liquid composition in varying amounts and diameters. In one embodiment, the amount of lanolin liquid composition that is applied to the nipple is approximately 140 μL or 0.14 grams (0.28 g for both nipples). In one embodiment, a sprayer may contain about 110 grams of lanolin liquid composition. In another embodiment, the amount of lanolin liquid composition that is applied is approximately 120 μL.

The lanolin liquid composition may be applied to the skin, particularly to the breasts, areolas, nipples, or combinations thereof, as often as needed. In addition, there are no usage limits. The skin may be exposed to the lanolin liquid composition for any duration of time. It is not necessary to clean the lanolin liquid composition off the breasts before breast feeding a baby.

### EXAMPLES

Exemplary embodiments of the present lanolin liquid composition are provided in the following examples. The following examples are presented to illustrate the present lanolin liquid composition and methods for applying the lanolin liquid composition to skin and to assist one of ordinary skill in making and using the same. The examples are not intended in any way to otherwise limit the scope of the invention.

### Prophetic Examples

### Example 1

The following lanolin liquid composition is manufactured by distilling raw farmed lanolin. The resulting distilled product is then filtered with 3× filtration, which reduces the pesticides to approximately 10 ppm or less. The filtering is followed by molecular distillation (steam), which reduces the pesticides to approximately 5 ppm or less. This step is followed by retaining the light oil fraction and removing all waxes, acids, heavy oils, and other undesirable components. This light oil fraction is finally diluted with oils. The dilution reduces the pesticides to approximately 0.7 ppm or less.

| Component | Amount (by weight) |
|---|---|
| Lanolin oil | 10.0% |
| Sunflower seed oil | 25.0% |
| Caprylic/capric triglycerides | 64.9% |
| Vitamin E acetate | 0.1% |

The above formulation is combined in a suitable homogenizer and processed for a period sufficient to produce a stable emulsion.

The above formulation is designed specifically for application onto skin, particularly the breasts, areolas, nipples, or combinations thereof. A sprayer is used to apply the lanolin liquid composition to the skin. A spray bottle that provides 900 sprays per bottle, or 0044/oz per spray is used. The spray bottle is selected to dispense a correct dosage of lanolin per spray, regardless of pump pressure. A relatively thin, even layer of the composition is applied to the skin. The volume applied and the area to which it is applied are selected to provide for rapid adsorption of the composition without rubbing, thereby avoiding or reducing additional irritation of the tissues, and without leaving a greasy film on the skin surface.

USP microbial counts are non-detect (less than 1) and pesticide concentrations are equivalent to or lower than USP modified lanolin.

### Comparative Example 1

USP modified lanolin is prepared by distilling raw farmed lanolin, which results in a high pesticide count. The distilled product is then subject to 3× filtration, which reduces pesticides to approximately 10 ppm or less. The filtered product is then subject to pesticide filtration, which reduces the pesticides to approximately 1 ppm or less.

What is claimed is:

1. A composition suitable for application to the skin comprising lanolin, an oil, an anti-oxidant, and a combination thereof.

2. The composition of claim 1, wherein the oil comprises sunflower seed oil or linoleic acid, canola oil, safflower oil, noni seed oil, corn oil, soy oil, evening primrose oil, pumpkin oil, wheat germ oil, rice bran oil, fractionated coconut oil or caprylic/capric triglycerides, and palm kernel oil, and combinations thereof.

3. The composition of claim 1, wherein the anti-oxidant comprises tocopherols and derivatives.

4. The composition of claim 1, further comprising a skin protectant, a humectant, an emollient, a skin conditioning agent, a skin care ingredient, a cosmetically active substance, a surfactant, and a combination thereof.

5. A composition comprising lanolin, sunflower seed oil, caprylic/capric triglycerides, tocopherol and derivatives, and a combination thereof.

6. The composition of claim 5, wherein the amount of lanolin in the composition may be from about 1% to about 30% by weight.

7. The composition of claim 5, wherein the amount of sunflower seed oil in the composition may be from about 1% to about 75% by weight.

8. The composition of claim 5, wherein the amount of caprylic/capric triglycerides in the composition may be from about 1% to about 90% by weight.

9. The composition of claim 5, wherein the amount of tocopherol and derivatives in the composition may be from about 0.01% to about 5% by weight.

10. A method for treating human skin, the method comprising:
spraying a composition comprising lanolin, an oil, an anti-oxidant, and a combination thereof on human skin.

11. The method of claim 10, wherein the oil comprises sunflower seed oil or linoleic acid, canola oil, safflower oil, noni seed oil, corn oil, soy oil, evening primrose oil, pumpkin oil, wheat germ oil, rice bran oil, fractionated coconut oil or caprylic/capric triglycerides, and palm kernel oil, and combinations thereof.

12. The method of claim 10, wherein the anti-oxidant comprises tocopherols and derivatives.

13. The method of claim 10, wherein the composition further comprises a skin protectant, a humectant, an emollient, a skin conditioning agent, a skin care ingredient, a cosmetically active substance, a surfactant, and a combination thereof.

14. A method for treating human skin, the method comprising:
applying a composition comprising lanolin, sunflower seed oil, caprylic/capric triglycerides, tocopherol and derivatives, and a combination thereof to human skin.

15. The method of claim 14, wherein the composition is applied using spraying technology.

16. The method of claim 14, wherein the composition is applied to a human breast.

17. The method of claim 15, wherein the composition is applied to a human areola, a nipple, and a combination thereof.

18. A method of preparing a composition comprising lanolin, an oil, an anti-oxidant, and a combination thereof, the method comprising:
filtering the composition one or more times and distilling the composition.

19. The method of claim 18, wherein distilling the composition comprises molecular distillation.

20. A method of preparing a composition comprising lanolin, the method comprising:
distilling raw farmed lanolin;
filtering distilled raw farmed lanolin with 3× filtration to produce filtered lanolin;
distilling the filtered lanolin using molecular distillation;
retaining a light oil fraction of lanolin and removing waxes, acids, heavy oils, and other undesirable components; and
diluting the light oil fraction of lanolin with oils.
